(19) United States
(12) Patent Application Publication
(10) Pub. No.: US 2018/0116947 A1
(43) Pub. Date: May 3, 2018

(54) WATER IN OIL EMULSION PROVIDING SKIN MATTITY AND TRUE COLOR

(71) Applicant: L'ORÉAL, Paris (FR)

(72) Inventors: Shauna-Kaye Lecaine Dominique SCOTLAND, Morristown, NJ (US); Balanda ATIS, Newark, NJ (US)

(73) Assignee: L'ORÉAL, Paris (FR)

(21) Appl. No.: 15/799,799

(22) Filed: Oct. 31, 2017

Related U.S. Application Data
(60) Provisional application No. 62/415,100, filed on Oct. 31, 2016.

(51) Int. Cl. A61K 8/891; A61K 8/25; A61K 8/81; A61K 8/88; A61K 8/06; A61Q 1/02

(52) U.S. Cl. CPC A61K 8/891; A61K 8/25; A61K 8/8152; A61K 2800/43; A61K 8/8111; A61K 8/064; A61Q 1/02; A61K 8/88

(57) ABSTRACT

The present invention relates to a water-in-oil cosmetic composition containing at least one hydrophobically treated powder, at least two mattifying powders, at least one silicone resin, and at least one oil absorbing powder, which impart true color and mattity, while also reducing dullness, whiteness, and/or ashiness of the keratinous substrate, in particular skin, to which they have been applied.

### CROSS-REFERENCE TO RELATED APPLICATIONS

This application claims the benefit of priority to U.S. provisional application no. 62/415,100 filed Oct. 31, 2016.

### TECHNICAL FIELD

The present invention relates to a water-in-oil cosmetic composition having at least one hydrophobically treated powder and at least two mattifying powders. Cosmetic compositions according to the invention impart true color and prolonged mattity, while also reducing dullness, whiteness, and/or ashiness of the keratinous substrate to which they have been applied, in particular skin. These compositions are particularly useful as foundations for deeper (darker) skin tones.

### BACKGROUND OF THE INVENTION

Foundations are known and used in the cosmetic field to impart even color to the skin. It is also known that foundations can help to control oil and shine on the skin. However, when preparing foundations for deeper ethnic skin tones, and especially in the case of oil-absorbing and/or matte compositions, it has been found that such compositions tend to impart an undesirable, dull or ashy hue to darker skin. It is related to the whitening effect of fillers that are typically used to overcome opaqueness of the cosmetic compositions.

Polymeric and mineral fillers as well as starches are well-known in the cosmetic field. Such fillers and starches are generally incorporated into cosmetic compositions as oil-absorptive components which tend to have good adherence to the skin and contribute to the matte properties of the compositions. However, the use of fillers and starches in compositions can tend to give the user an undesirable ashy and/or dull appearance and may, in some cases, result in a dry skin feel. Moreover, the amount of fillers/starch necessary to adequately absorb oil and prevent shine generally also tends to whiten the formulation and give the ashy dull hue that becomes more apparent after the composition dries on the skin. In addition, the color of foundations applied to the skin changes over time and does not represent the initial shade. These problems are greatly exacerbated in the case of foundations for women with deeper ethnic skin tones.

Thus, there remains a need in the cosmetic industry to provide consumers having deeper ethnic skin tones with cosmetic products having improved properties such as improved oil absorption, shine reduction, wear of mattity, matte, texture, long-wear properties, and/or ability to impart true color, while also reducing dullness, whiteness, and/or ashiness of the skin.

The current inventive compositions are water-in-oil emulsions containing at least one hydrophobically treated powder, for example, pigments, as well as other powders providing enhanced oil absorption, long mattity, shine control and long wear. The compositions have good product application and coverage, and quickly transition to a powder upon application to a keratinous material, such as skin, retaining oil and shine-control properties.

### SUMMARY OF THE INVENTION

The present invention relates to a water-in-oil emulsion which is long wearing, while at the same time, provides long term mattity and true color, containing:
a) at least one hydrophobically treated powder;
b) at least two mattifying powders;
c) at least one silicone resin;
d) at least one oil absorbing powder;
e) optionally at least one adhesive agent; and
f) optionally at least one silicone elastomer, by weight, relative to the total weight of the composition.

The present invention further relates to methods for making up a keratinous substrate, preferably skin, comprising applying to the keratinous substrate a composition according to the invention.

The present invention further relates to the above water-in-oil emulsion containing at least two mattifying powders (b), wherein the ratio of a first mattifying powder to a second mattifying powder is greater than from about 0.10:1 and lower than from about 1:10, by weight, relative to the total weight of the composition, preferably from about 2:1 and lower than from about 1:10.

The present invention further relates to a water-in-oil emulsion comprising:
a) from about 0.05% to about 80% of at least one hydrophobically treated powder;
b) from about 0.05% to about 15% of at least two mattifying powders;
c) from about 1% to about 30% of at least one siloxane resin;
d) from about 0.5% to about 20% of at least one oil absorbing powder;
e) optionally from about 0.5% to about 6% of at least one adhesive agent; and
f) optionally from about 1% to about 30% of at least one silicone elastomer, by weight, relative to the total weight of the composition.

The present invention further relates to the above water-in-oil emulsion containing a water phase, wherein the ratio of the water phase to hydrophobically treated powder is preferably lower than from about 4:1, preferably lower than from about 3:1, preferably lower than from about 2:1, preferably lower than from about 1.5:1.

The present invention further relates to the above water-in-oil emulsion in the form of foundations, blushes, liners, eyeshadows, skin care products, or sunscreens.

The present invention further relates to methods of making also relates to a method of making a water-in-oil emulsion which is long wearing, while at the same time, provides long term mattity and true color, by combining:
a) at least one hydrophobically treated powder;
b) at least two mattifying powders;
c) at least one silicone resin ;
d) at least one oil absorbing powder;
e) optionally at least one adhesive agent; and
f) optionally at least one silicone elastomer, by weight, relative to the total weight of the composition, to form an water-in-oil emulsion.

It is to be understood that both the foregoing general description and the following detailed description are exemplary and explanatory only, and are not restrictive of the invention.

### DETAILED DESCRIPTION OF THE INVENTION

The aim of the present invention is to provide a water-in-oil emulsion which has good stability, while at the same time retaining good cosmetic properties. In particular, it is desired to provide a stable water-in-oil emulsion comprising at least one hydrophobically treated powder, pigments in particular, which are more typically used in oil-in-water emulsions.

The cosmetic compositions of the present invention are preferably water-in-oil emulsions, which contain low amount of water (less than about 30%) and have a high powder load (greater than 10% total powders). These compositions provide a unique texture in that they are initially creamy and light, affording good spreadability (increased product pick up and coverage) and also feel less drying. The compositions impart true and over time unvarying color while reducing the appearance of ashiness, whiteness, and/or dullness of the skin, which is especially useful for individuals having deeper ethnic skin tones. In addition, the inventive compositions have lasting mattifying effect.

These compositions are stable at accelerated conditions, as well as they have a long shelf life (stable longer than two (2) months).

In the following description of the invention and the claims appended hereto, it is to be understood that the terms used have their ordinary and accustomed meanings in the art, unless otherwise specified.

It is also to be understood that, as used herein the terms “the,” “a,” or “an,” mean “at least one,” and should not be limited to “only one” unless explicitly indicated to the contrary. Thus, for example, the use of “an oil absorbing powder” is intended to mean at least one oil absorbing powder.

“About” as used herein means within 10% of the indicated number (e.g. “about 10%” means 9%-11% and “about 2%” means 1.8%-2.2%).

The terms “ashy,” “ashiness” and variations thereof are intended to refer to the appearance of a gray overcast on the skin or keratinous substrate after the product has been applied and evaluated after drying.

The term “deeper ethnic skin tones” is intended to refer to those skin tones that are darker and tend to have more pronounced and deeper red, red/yellow, and yellow undertones. Users with deeper ethnic skin tones may belong to ethnicities including, but not limited to, African, African-American, Caribbean, Hispanic, Middle Eastern, and Indian ethnicities.

“Free” or “devoid” means that while it is preferred that no amount of the specific component be present in the composition, it is possible to have very small amounts of it in the compositions of the invention provided that these amounts do not materially affect at least one, preferably most, of the advantageous properties of the compositions of the invention. Thus, for example, “free of X” means that X is preferably omitted (that is 0% by weight), but can be present in the composition at an amount of less than about 0.25% by weight, typically less than about 0.1% by weight, typically less than about 0.05% by weight, based on the total weight of the composition as a whole.

“Liquid” or “liquid cosmetic” or “liquid foundation” or “liquid composition” means a composition having a fixed volume, flows to cover the bottom and assumes the shape of the portion of the container it fills and is slightly compressible (as disclosed in General chemistry, Fourth Edition 2005, p.434.

“Water-in-oil emulsion” means a colloidal dispersion in which small droplets of the water phase (discontinuous phase) are dispersed within the oil phase (continuous phase).

“Matte”, as used in foundation means that the composition is not shiny, shimmery or wet looking. Rather, it is powdery looking. “Good wear of mattity” or “mattity” refers to a product/composition's ability to maintain a matte skin appearance and/or feel throughout a period of time, such as a day or the consumer's normal wear time for the product.

The term “true color” is intended to refer to a color or shade that is most accurate or closest to the tone of the keratinous substrate to which it is applied. For instance, in at least certain exemplary embodiments, a composition that imparts true color according to the disclosure closely matches the skin tone without leaving an ashy, dull, white, and/or gray appearance. True color refers to the color obtained not only upon application to the keratinous substrate, but also after drying and wearing the composition over time (from least about 4 hours up to about 24 hours of wear).

“Long wear” compositions as used herein, refers to compositions where color remains the same or substantially the same as at the time of application, as viewed by the naked eye, after an extended period of time. Long wear properties may be evaluated by any method known in the art for evaluating such properties. For example, long wear may be evaluated by a test involving the application of a composition to human hair, skin or lips and evaluating the color of the composition after an extended period of time. For example, the color of a composition may be evaluated immediately following application to hair, skin or lips and these characteristics may then be re-evaluated and compared after a certain amount of time. Further, these characteristics may be evaluated with respect to other compositions, such as commercially available compositions. For foundation compositions, “long wear” typically means the composition remains on the skin at least about 4 hours up to about 24 hours, and retains rich color.

“Substituted” as used herein, means comprising at least one substituent. Non-limiting examples of substituents include atoms, such as oxygen atoms and nitrogen atoms, as well as functional groups, such as amine groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphategroups, siloxane groups, and polysiloxane groups. The substituent(s) may be further substituted.

“Stable” or “stable composition” or “stable emulsion” or “stable water-in-oil” emulsion” means that the composition does not change its physical properties (e.g., separation of water and oil phases, creaming, pigment sedimentation) over time and/or under challenging conditions (high or low temperature).

“Comprising” it is meant that other steps and/or ingredients which do not affect the end result may be added. The products, compositions, methods and processes of the present invention can include all the essential elements and limitations of the invention described herein as well as any of the additional or optional ingredients, components, steps, or limitations described herein.

As used herein, all ranges provided are meant to include every specific range within, and combination of subranges between, the given ranges. Thus, a range from 1-5, includes specifically 1, 2, 3, 4 and 5, as well as subranges such as and 2-5, 3-5, 2-3, 2-4, 1-4, etc.

Unless otherwise specified herein, all percentages and ratios of components are by weight relative to the total weight of the final composition.

Hydrophobically Treated Powders

According to the present invention, water-in-oil emulsions comprising at least one hydrophobically treated powder are provided. Powders suitable for use in the present invention include, but are not limited to, hydrophobically treated: water-soluble dyes, fat-soluble dyes, inorganic pigments, pearlescent agents, lakes, fillers and their mixtures.

These coloring materials can be treated at the surface with a hydrophobic agent, such as silanes, silicones, fatty acid soaps, C₉₋₁₅ fluoroalcohol phosphates, acrylate/dimethicone copolymers, mixed C₉-C₁₅ fluoroalcohol phosphate/silicone copolymers, lecithins or hydrogenated lecithin, carnauba wax, polyethylene, chitosan and optionally acylated amino acids, such as lauroyl lysine, disodium stearoyl glutamate and aluminium acyl glutamate.

In at least one embodiment, the surface-treated pigments and other powders that are useful in the context of the present disclosure may also be treated with a mixture of these compounds and/or may have undergone several surface treatments.

The surface-treated pigments and fillers that are useful in the context of the present disclosure may be prepared according to surface-treatment techniques that are known to those skilled in the art, or may be commercially available in the required form.

According to one embodiment of the present disclosure, the surface-treated pigments and/or powders are coated with at least one organic layer.

The at least one organic agent with which the pigments are treated may be deposited on the pigments or fillers by any known method, for example, evaporation of solvent, chemical reaction between the molecules of the surface agent or creation of a covalent bond between the surface agent and the pigments.

The surface treatment may thus be performed, for example, by chemical reaction of at least one surface agent with the surface of the pigments or fillers and creation of a covalent bond between the at least one surface agent and the pigments or powders. This method is described, for example, in U.S. Pat. No. 4,578,266.

In at least one embodiment, at least one organic agent covalently bonded to the pigments or powder may be used.

The at least one agent for the surface treatment may be present in an amount ranging from 0.1 percent to 50 percent by weight, for example, from 0.5 percent to 30 percent by weight, or from 1 percent to 10 percent by weight relative to the total weight of the surface-treated pigments or powder.

According to one embodiment of the present disclosure, the at least one surface treatment of the pigments and fillers may be chosen from the following treatments:

PEG-silicone treatments, for instance the AQ surface treatment sold by LCW;

chitosan treatments, for instance the CTS surface treatment sold by LCW;

triethoxycaprylylsilane treatments, for instance the AS surface treatment sold by LCW;

methicone treatments, for instance the SI surface treatment sold by LCW;

dimethicone treatments, for instance the Covasil 3.05 surface treatment sold by LCW;

dimethicone/trimethyl siloxysilicate treatments, for instance the Covasil 4.05 surface treatment sold by LCW;

lauroyllysine treatments, for instance the LL surface treatment sold by LCW;

lauroyllysine dimethicone treatments, for instance the LL/SI surface treatment sold by LCW; magnesium myristate treatments, for instance the MM surface treatment sold by LCW;

aluminium dimyristate treatments, for instance the MI surface treatment sold by Miyoshi;

perfluoropolymethyl isopropyl ether treatments, for instance the FHC surface treatment sold by LCW;

isostearyl sebacate treatments, for instance the HS surface treatment sold by Miyoshi;

disodium stearoyl glutamate treatments, for instance the NAI surface treatment sold by Miyoshi;

dimethicone/disodium stearoyl glutamate treatments, for instance the SA/NAI surface treatment sold by Miyoshi;

perfluoroalkyl phosphate treatments, for instance the PF surface treatment sold by Daito;

acrylate/dimethicone copolymer and perfluoroalkyl phosphate treatments, for instance the FSA treatment sold by Daito;

polymethylhydrogenosiloxane/perfluoroalkyl phosphate treatments, for instance the FS01 surface treatment sold by Daito;

lauroyllysine/aluminium tristearate treatments, for instance the LL-AlSt surface treatment sold by Daito;

octyltriethylsilane treatments, for instance the OTS surface treatment sold by Daito;

octyltriethylsilane/perfluoroalkyl phosphate treatments, for instance the FOTS surface treatment sold by Daito;

acrylate/dimethicone copolymer treatments, for instance the ASC surface treatment sold by Daito;

isopropyl titanium triisostearate treatments, for instance the ITT surface treatment sold by Daito;

microcrystalline cellulose and carboxymethylcellulose treatments, for instance the AC surface treatment sold by Daito;

cellulose treatments, for instance the C2 surface treatment sold by Daito;

acrylate copolymer treatments, for instance the APD surface treatment sold by Daito; and

perfluoroalkyl phosphate/isopropyl titanium triisostearate treatments, for instance the PF+ITT surface treatment sold by Daito.

According to preferred embodiments of the present invention, hydrophobically treated powders, including hydrophobically pigments, are incorporated into the oil phase of the inventive composition.

The at least one surface-treated pigment may be present in the inventive composition in total amounts generally ranging from 0.05 percent to 50 percent by weight, for example, from 0.1 percent to 35 percent by weight, or from 0.5 percent to 20 percent by weight relative to the total weight of the composition.

The at least one surface-treated powder may be present in the composition in total amounts ranging from 0.05 percent to 80 percent by weight, for example, from 0.1 percent to 50 percent by weight, or from 0.5 percent to 30 percent by weight relative to the total weight of the composition.

According to at least one embodiment, the composition in accordance with the present disclosure may further comprise at least one surface-untreated filler.

In another embodiment, the composition in accordance with the present disclosure may further comprise at least one surface-untreated pigment.

Mattifying Powders

In accordance with the present invention, compositions comprising at least two mattifying powders are provided.

Examples of mattifying powders that may be used in the compositions of the invention are mineral mattifiers. These mattifiers can include, for example, lamellar or nonlamellar, colorless or white mineral particles of any shape, such as spherical, platelet, or oblong particles.

Advantageously, the mattifying polymeric powders are sebum-absorbing particles characterized by having a sebum uptake of greater than or equal to 10 ml/100 g, especially greater than or equal to 20 ml/100 g and in particular greater than or equal to 30 ml/100 g, and preferably greater than about 70 ml/100 g. The term “sebum-absorbing particle” means a powder that is capable of absorbing and/or adsorbing sebum. Generally, this type of particle is in the form of a powder of particles having a sebum uptake.

The sebum uptake corresponds to the amount of sebum absorbed and/or adsorbed by the particle. It is measured according to the wet point method described below.

Advantageously, the sebum-absorbing particle may have a BET (Brunauer-Emmett-Teller) specific surface area of greater than or equal to 200 m²/g, preferably greater than 350 m²/g and preferentially greater than 500 m²/g, and especially less than 2000 m²/g.

The BET specific surface area is determined according to the BET (Brunauer-Emmett-Teller) method described in The Journal of the American Chemical Society, Vol. 60, page 309, February 1938 and corresponding to the international standard ISO 5794/1 (appendix D). The BET specific surface area corresponds to the total specific surface area (thus including micropores) of the particle and especially of the powder.

The mattifying powders (sebum-absorbing particles) may be an inorganic powders or an organic powders. The particle or particles of one or more inorganic compounds used in the cosmetic composition may have different shapes, for example regular or irregular spheres, full or hollow, glitter, needles or platelets and preferably they are approximately spherical. The useful mattifying agent are described for example in US2015/174044, the disclosures of which is hereby incorporated by reference.

In certain embodiments, the mineral mattifiers may be chosen from mica, hectorite, calcium aluminum borosilicate, precipitated calcium carbonate, magnesium carbonate, magnesium hydrogen carbonate, hydroxyapatite, hollow silica microspheres, glass or ceramic microcapsules, clay, quartz, natural diamond powder, and mixtures thereof.

As per invention, the mattifying powders can be also chosen from acrylic polymer powders. Such powders include polymethylsilsesquioxane, methacrylate polymers, for example methyl methacrylate/glycol dimethacrylate crosspolymer, methyl methacrylate crosspolymer, polymethyl methacrylate powders, polymethyl methacrylate/ethylene glycol dimethacrylate powders, polyallyl methacrylate/ethylene glycol dimethacrylate powders, and ethylene glycol dimethacrylate/lauryl methacrylate copolymer powders, and mixtures thereof.

Commercial examples of acrylic polymer powder products include methacrylate polymers such as polymethylsilsesquioxane sold under the name Tospearl® 150KA polymethyl methacrylate powders sold under the name Covabead® LH85 by Wacker, DSPCS-12 series and SPCAT-12 from Kobo, Poly-Pore 200 series from Amcol, and Techpolymer MBP-8 (methyl methacrylated crosspolymer) from Sekisui Plastics, SEPIMAT™ from Seppic and Matsumoto Microsphere®M-series available from Matsumoto Yushi-Seiyaku; the polymethyl methacrylate/ethylene glycol dimethacrylate powders sold under the names Microsponge® 5640 Skin Oil Adsorber (methyl methacrylate/glycol dimethacrylate crosspolymer, Dow Corning) and Ganzpearl® GMP-0820 (Ganz Chemical); the polyallyl methacrylate/ethylene glycol dimethacrylate powders sold under the name Poly-Pore® L200 and Poly-Pore® E200 (Amcol); and acrylic acid copolymers available from Dow Corning/Enhanced Derm Technologies under the name Polytrap® (for example ethylene glycol dimethacrylate/lauryl methacrylate copolymer powder, sold under the name Polytrap® 6603).

Examples of suitable acrylic polymer powders are provide in US2002/018791 and US2014/227213, the disclosers of which are hereby incorporated by references.

According to another embodiment, the compositions of the present invention include polylactic acid based mattifying agent, such as for example described in U.S. Pat No. 8,968,787 and U.S. Pat. No. 9,017,812, the disclosures of which are hereby incorporated by references. The inventive compositions incorporate polylactic acid powders having particles size smaller than about 100 microns, preferably smaller than from about 50 microns and larger than about 2 microns. They are commercially available from Toray Industries as Toraypearl™ series of polymer polylactic particles.

According to the present invention, the mattifying powders are present in the inventive cosmetic compositions in an amount ranging from about 0.05% to about 15% by weight, for example, from about 1% to about 10%, or from about 5% to about 8%, by weight, relative to the total weight of the composition, including all ranges and subranges therebetween.

In various embodiments, the mattifiers are chosen from silica powders, calcium sodium borosilicate, polylactic acid, methyl methacrylate crosspolymer powders and mixtures thereof.

The mattifying powders useful in the present invention are incorporated into the oil phase.

According to one exemplary embodiment the inventive composition contains at least two mattifying powders, wherein the ratio of one mattifying powder to a second mattifying powder is from about 0.1:1 to about 1.5: 1.

Silicone Resin

According to the present invention, compositions comprising at least one silicone resin are provided. Suitable silicone resins include, but are not limited to, silicone resins such as described for example, in U.S. Pat. No. 5,505,937, U.S. Pat. No. 5,911,974, U.S. Pat. No. 5,965,112, U.S. Pat. No. 5,985,298, U.S. Pat. No. 6,074,654, U.S. Pat. No. 6,780,422, U.S. Pat. No. 6,908,621, the disclosures of which are hereby incorporated by references.

According to preferred embodiments, the cosmetic compositions may contain siloxysilicate resins. One non-limiting example of a siloxysilicate in accordance with the present invention is trimethylsiloxysilicate, which may be represented by the following formula:

[(CH₃)₃SiO]ₓ(SiO_{4/2})_{y}

wherein x and y may, for example, range from 50 to 80. Such siloxysilicates are commercially available from General Electric, Dow Corning, Wacker, Milliken, Siltech, Grant Industries, Momentive and Shin-Etsu Silicones under the tradename Resin MQ®.

According to another embodiment of this invention, the compositions may contain silsesquioxane resins, including comprise at least one polypropyl silsesquioxane film forming resin.

Silsesquioxane resins are a specific form of silicone resin. Silicone resin nomenclature is known in the art as “MDTQ” nomenclature, whereby a silicone resin is described according to the various monomeric siloxane units which make up the polymer. Each letter of “MDTQ” denotes a different type of unit. When the film forming resin is made up predominantly of tri-functional units (or T units), it is generally called a silsesquioxane resin, which is described, for example in US 2006/0292096, herein incorporated by reference. Optionally, the units can contain modifications containing long chain carbons such as, for example, C9-C75 carbon chains, C16-C60 carbon chains, C20-C50 carbon chains, C30-C45 carbon chains, etc. The resulting modified resin is sometimes referred to as a “resin wax.”

Examples of silsesquioxane resins that may be used in the present invention are alkyl silsesquioxane resins that are silsesquioxane homopolymers and/or copolymers having an average siloxane unit of the general formula R¹ₙ SiO_{(4-n)/2}, wherein each R1 is a propyl group, wherein more than 80 mole % of R1 represent a C3-C10 alkyl group, n is a value of from 1.0 to 1.4, and more than 60 mole % of the copolymer comprises R¹SiO_{3/2} units. As each R1 is a propyl group these polymers are called polypropylsilsesquioxane resins or “t-propyl” silsesquioxane resins. These resins and methods of making them are described, for example in U.S. Pat. No. 8,586,013, US2012/0301415, US2007/0093619, and US2006/0292096, all of which are herein incorporated by reference.

A non-limiting example of a polypropylsilsesquioxane resin suitable for use in the present invention is commercially available from Dow Corning as “DC 670” Fluid or “DC 680” Fluid. These Dow Corning resins have a general formula of RₙSiO_{(4-n)/2} wherein R is independently chosen from a hydrogen atom and a monovalent hydrocarbon group comprising 3 carbon atoms, wherein more than 80 mole % of R are propyl groups, n is a value from 1.0 to 1.4, more than 60 mole % of the copolymer comprises RSiO_{3/2} units, and having a hydroxyl or alkoxy content from 0.2 to 10% by weight, for example between 1 and 4% by weight, preferably between 5 and 10% by weight, and more preferably between 6 and 8% by weight. Preferably, the polypropylsilsesquioxane resin has a molecular weight from about 5000 to about 30,000 and a Tg from about —5° C. to about 5° C. Another example of a suitable silicone resin is C30-45 alkyldimethylsilyl propylsilsesquioxane (and) paraffin, a silicone resin in which units contain a long chain carbon.

Another embodiment of this invention, exemplifies the composition containing at least one siloxysilicate resin, at least one silsesquioxane resin and/or mixture thereof.

The at least one silicone resin is generally present in the cosmetic composition of the present invention in an amount ranging from about 1% to about 30% by weight; such as from about 1% to about 20% by weight; such as from about 2% to about 15% by weight, all weights being based on the weight of the composition as a whole.

As per another embodiment, the compositions of the present invention may contain the mixture of MQ and T propyl silicone resins in a dry form, as described for example in U.S. Pat. No. 7,803,358, U.S. Pat. No. 9,023,335, U.S. Pat. No. 8,124,710 and U.S. Pat. No. 8,604,154, all of which are herein incorporated by reference.

In the embodiment, the silicone resin is the mixture of MQ and T propyl silicone resins obtained by mixing:

A) MQ resin comprising at least 80 mole % of siloxy units selected from

(R¹₃SiO_{1/2})ₐ and (SiO_{4/2})_{b} units, where R¹ is independently an alkyl group having from 1 to 8 carbon atoms, an aryl group, a carbinol group, or an amino group, with the proviso that at least 95 mole % of the R¹ groups are alkyl groups, a and b has a value greater than zero, and the ratio of a/b is 0.5 to 1.5; and

B) a propyl silsesquioxane resin comprising at least 80 mole % R³SiO_{3/2} units, where R³ is independently an alkyl group having from 1 to 8 carbon atoms, an aryl group, a carbinol group, or an amino group, with the proviso that at least 40 mole % of the R³ groups are propyl.

The ratio the weight ratio of component A to B is from 90:10 to 10:90, usefully the A/B ratio is equal to 85:15 or 50:50 or 65: 35 or 30:70.

A non-limiting example of siloxane resins resin suitable for use in the present invention is commercially available from Dow Corning under trade name “DC MQ-1640”.

According to the invention, the amount of silicone resin, by weight of active material (dry matter), will advantageously range from about 5% to about 30% by weight; such as from about 10% to about 25% by weight; such as from about 15% to about 20% by weight, all weights being based on the weight of the composition as a whole.

Oil absorbing powder

In accordance with the present invention, compositions comprising at least one oil absorbing powder are provided. “Oil absorbing powders” mean powders that help absorb an excess sebum and help set up a makeup so it is not easily removable under high humidity conditions (e.g., temperature higher than 20° C. and relative humidity higher than 40%). The oil absorbing powders may be an inorganic powder or an organic powder. The particle or particles of one or more inorganic compounds used in the cosmetic composition may have different shapes, for example spheres, full or hollow, glitter, needles or platelets and preferably they are approximately spherical. The useful oil absorbing powders are described for example in U.S. Pat. No. 5,035, 885 and U.S. Pat. No. 9,308,396, the disclosures of which are hereby incorporated by references. The oil absorbing powders useful in this invention are different than described overhead the “mattifying powders”.

Examples of oil absorbing powders that may be used in the compositions of the invention include, for example, silica powder; talc; polyamide particles and especially those sold under the name Orgasol by the company Atochem; polyethylene powders; microspheres based on acrylic copolymers, such as those based on ethylene glycol dimethacrylate/lauryl methacrylate copolymer sold by the company Dow Corning under the name Polytrap; expanded powders such as hollow microspheres and especially the microspheres sold under the name Expancel by the company Kemanord Plast or under the name Micropearl F 80 ED by the company Matsumoto; powders of natural organic materials such as crosslinked or noncrosslinked corn starch, wheat starch or rice starch, such as the powders of starch crosslinked with octenyl succinate anhydride, sold under the name Dry-Flo by the company National Starch; silicone resin microbeads such as those sold under the name Tospearl by the company Toshiba Silicone; clays (bentone, laponite, saponite, etc.); and mixtures thereof. The examples of oil absorbing powders are disclosed for example in US2015/174044, disclosure of which is hereby incorporated by reference.

Non-limiting examples of commercially available silica powders include the porous silica microspheres sold under the name Sunsphere® H51 and H33 by Asahi Glass, aerogel sold under the name VM-2260 (INCI name: Silica silylate) by Dow Corning and the polydimethylsiloxane-coated amorphous silica microspheres sold under the names SA Sunsphere® H 33 and H53 by Asahi Glass.

Non-limiting examples further include an aluminum-based starch as a non-limiting example of the oil absorbing powders. Preferably, the starch is a modified starch.

Modified starches are generally described in Kirk-Othmer Encyclopedia of Chemical Technology, Fourth Edition, Volume 22, pp.699-719. Modified starches useful in cosmetics are also described in U.S. Pat. No. 6,294,180 and U.S. Pat. No. 6,416,751, incorporated herein by reference.

Non-limiting examples of aluminum-based modified starches that can be used in the invention include starches esterified with octenylsuccinic anhydride, and particularly aluminum starch octenyl succinate, such as the products under the tradenames Natrosorb® W, Natrosorb® HFB and Natrosorb® HFW, and DryFlo, such as DryFlo® PC, DryFlo® plus and DryFlo® AF pure (all from Akzo Nobel (National Starch)), as well as the product Agenaflo® OS 9051 from Agrana Starch.

The aluminum-based starch is present in the compositions of the invention in preferred amounts generally ranging from about 20% to about 35% by weight, typically from about 22% to about 33% by weight, more typically from about 25% to about 30% by we, weight, based on the total weight of the composition, including all ranges and subranges in between.

In certain embodiments, the oil absorbing powders may be chosen from polyamide powders, such as Nylon® or Orgasol® powders from Arkema; cellulose poly-β-alanine and polyethylene powders; tetrafluoroethylene polymer powders, such as Teflon® powders; lauroyllysine; polymeric hollow microspheres such as those of polyvinylidene chloride/acrylonitrile, for instance Expancel® from Nobel Industries; silicone resin microbeads, such as Tospearls® from Toshiba; elastomeric polyorganosiloxane particles, such as those obtained by polymerization of organopolysiloxane having at least two hydrogen atoms each bonded to a silicon atom and of an organopolysiloxane comprising at least two ethylenically unsaturated groups, for instance, two vinyl groups, in the presence of a platinum catalyst; and metal soaps derived from organic carboxylic acids comprising from 8 to 22 carbon atoms, such as from 12 to 18 carbon atoms, for example, zinc stearate, magnesium stearate, lithium stearate, zinc laurate, and magnesium myristate.

According to various embodiments, the mineral oil absorbing powders may be chosen from silica based powders, including calcium sodium borosilicate, and mixtures thereof. An example of a commercial calcium sodium borosilicate is Luxsil Cosmetic Spheres available from Potters Industry.

The oil absorbing powders are preferably present in an amount of from about 0.5 percent to about 20 percent by weight, such as from about 1 percent to about 10 percent by weight, such as from about 3 percent to about 5 percent by weight, including all ranges and subranges there between, all weights being based on the total weight of the composition.

Adhesive Powder (Optional)

According to another embodiment, a composition according to the invention may further contain at least one adhesive powder. The adhesive powders may be chosen for example from organopolysiloxane powders coated with silicone, silsesquioxane powders, polymethylsilsesquioxane powders in particular and the mixtures thereof.

Polymethylsilsesquioxane powders that may especially be useful for this invention include silicone resin microbeads, such as those sold under the name Tospearl by the company Momentive Performance Materials, and especially under the reference Tospearl 120A, 130A, CF600, 145A, 150KA and 2000B. These resins are spherical silicone resins available in 2.0, 3.0, 4.5, 4.5, 5.0 and 6.0 micron average median particle sizes, respectively. Tospearl 3120 is available with a 13.0 micron average median particle size.

The at least one adhesive powder may, in various exemplary embodiments, be present in the cosmetic compositions in an amount ranging from about 0.5% to about 6% by weight, for example, from about 0.7% to about 4%, or from about 1.0% to about 3%, relative to the total weight of the composition, including all ranges and subranges therebetween.

Silicone Elastomer (Optional)

According to preferred embodiments, the composition according to the invention further comprises at least one silicone elastomer.

In a preferred embodiment, the composition comprises a non-emulsifying silicon elastomer.

The non-emulsifying silicon elastomer may be in the form of a gel or a powder.

The “organopolysiloxane elastomer” or “silicone elastomer” or “silicone crosspolymer” thickens the composition, adds the cushiony (spongy) effect and improves the application of the finished product. Also, it provides a very soft feel and contributes to the prolonged mattifying effect of the inventive composition after the application, which is especially advantageous for skin products.

The term “non-emulsifying” defines organopolysiloxane elastomers that do not contain in any hydrophilic chains, and in particular polyoxyalkylene (especially polyoxyethylene or polyoxypropylene) or polyglyceryl units. Thus, according to one particular embodiment of the invention, the composition comprises an organopolysiloxane elastomer that is free of polyoxyalkylene units and polyglyceryl units.

The non-emulsifying elastomers are described in US. Pat. No. 8,637,057, the disclosure of which is hereby incorporated by reference.

The non-emulsifying elastomers particularly useful in this invention include but not limit those sold under the names KSG-6, KSG-15, KSG-16, KSG-18, KSG-41, KSG-42, KSG-43 and KSG-44 by the company Shin-Etsu, DC 9040, DC 9041, DC 9509, DC 9505 and DC 9506, by the company Dow Corning, and SFE 839 by Momentive Performance Material.

In an embodiment, the organopolysiloxane elastomer particles are conveyed in the form of a gel formed from an elastomeric organopolysiloxane included in at least one hydrocarbon-based oil and/or one silicone oil. In these gels, the organopolysiloxane particles are often non-spherical particles.

Not limited examples of silicone elastomers useful in this invention are dimethicone crosspolymer gels (blends of dimethicone crosspolymers in solvents) having viscosity values from about 150 and to about 700 mm²/s, from about 200 to about 650 mm²/s and from about 300 to about 600 mm²/s.

Particularly useful for this invention may be blends of high molecular weight silicone elastomers in volatile solvents, such as silicone oils, hydrocarbon oils and mixtures thereof, as per definition disclosed far along.

The specific but not limiting examples of silicone elastomeric gels applicable in this invention are represented by DC EL-8040 ID (INCI name: Isododecane (and) Dimethicone Crosspolymer) and DC EL-9140 DM (INCI name: Dimethicone (and) Dimethicone Crosspolymer), supplied by Dow Corning.

Non-limiting examples of silicone elastomers and their synthesis are disclosed, for example in U.S. Pat. No. 8, 637,057 and US/20150174048, all of which are herein incorporated by reference.

This silicon elastomers present in the inventive compositions are generally in a content ranging from 1 percent to 30 percent by weight of active material (dry matter), more preferably from about 1.5 percent to about 20 percent and most preferably from 2 percent to 10 percent by weight relative to the total weight of said composition.

Emulsifiers (Optional)

According to preferred embodiments, the compositions may further contain emulsifiers, preferably selected from liquid fatty esters, silicone based fatty emulsifiers and mixtures thereof. The emulsifiers are preferably present in the inventive compositions in amounts ranging from about 20% to about 50%.

Liquid Fatty Ester

The compositions of the invention may comprise one or more liquid fatty esters.

The term “liquid fatty ester” means an ester that that is liquid at room temperature and atmospheric pressure (25° C., 1 atm) and which comprises in its structure at least one hydrocarbon-based chain containing at least 6 carbon atoms. Preferably, it has a melting point of less than or equal to about 10° C.

The liquid fatty esters may be esters of monoalcohols or of polyols with monocarboxylic or polycarboxylic acids, at least one of the alcohols and/or acids comprising at least one hydrocarbon-based chain containing at least 6 carbon atoms. Such liquid fatty esters are described, for example in US2012276029.

Preferably, the liquid fatty ester according to the invention is chosen from esters of a fatty acid (at least 6 carbon atoms) and of a monoalcohol, more particularly from esters of a fatty monoacid and of a monoalcohol. Preferably, at least one of the alcohols and/or acids is branched. Preferably, the alcohol and/or the acid are saturated, and preferentially both are saturated. Preferentially, the liquid fatty ester is not oxyalkylenated.

The liquid fatty esters according to the invention are preferably of formula

R1-COOR2

(I)

in which:

R1 denotes a linear or branched, saturated or unsaturated, optionally mono- or polyhydroxylated hydrocarbon-based radical, containing from 5 to 31 carbon atoms, preferably containing from 7 to 21 carbon atoms, and

R2 denotes a linear or branched, saturated or unsaturated, optionally mono- or polyhydroxylated hydrocarbon-based radical, containing from 1 to 20 carbon atoms.

Preferably, R1 denotes a linear or branched alkyl (saturated) radical containing 7 to 21 carbon atoms, especially from 8 to 17 carbon atoms, and more preferably from 8 to 15 carbon atoms.

Preferably, R2 denotes a linear alkyl (saturated) radical containing 1 to 4 carbon atoms or a branched alkyl (saturated) radical containing from 3 to 20 carbon atoms, especially from 3 to 16 carbon atoms. More preferably, R2 denotes a branched saturated alkyl radical containing from 3 to 12 carbon atoms.

Non-limiting examples of monoesters of monoacids and of monoalcohols useful in the invention include ethyl laurate, butyl laurate, hexyl laurate, isohexyl laurate, isopropyl laurate, isoamyl laurate, methyl myristate, ethyl myristate, butyl myristate, isobutyl myristate, isopropyl myristate, 2-octyldodecyl myristate, 2-ethylhexyl monococoate (or octyl monococoate), 2-ethylhexyl isononanoate, ethyl palmitate, isopropyl palmitate, isobutyl palmitate, 2-ethylhexyl palmitate (or octyl palmitate), butyl stearate, isopropyl stearate, isobutyl stearate, isocetyl stearate, isostearyl isostearate, isopropyl isostearate, 2-ethylhexyl stearate (or octyl stearate), 2-ethylhexyl hydroxystearate (or octyl hydroxystearate), decyl oleate, isononyl isononanoate, isodecyl neopentanoate, tridecyl neopentanoate, isocetyl neopentanoate, isostearyl neopentanoate, octyldodecyl neopentanoate and isoarachidyl neopentanoate, and mixtures thereof.

Preferably, the liquid fatty ester used in the invention is chosen from isodecyl neopentanoate, isononyl isononanoate, tridecyl neopentanoate, and mixtures thereof.

The compositions according to the invention may comprise the liquid fatty ester in an amount preferably ranging from 0.1% to 15% by weight, from 5% to 12% by weight, preferentially from % to % by weight and better still from 7% to 10% by weight, relative to the total weight of the composition, and including all ranges and sub ranges there between.

Organosilicone Emulsifiers

Silicone based fatty solvents are particularly useful, particularly in embodiments wherein the emulsion is a water-in-oil (water-in-silicone) emulsion. Such emulsifiers include silicone polyethers and polyalkoxylated silicones.

Silicone polyethers contain oxyethylene units of the formula CH₂CH₂O, e.g., such as described in U.S. Pat. No. 4,268,499, U.S. Patent Application Publication No. 2002/0028223 and U.S. Patent Application Publication No. 2003/0049212, all of which are herein incorporated by reference.

Examples of silicone polyethers include PEG/PPG-18/18 Dimethicone, available as a blend with cyclopentasiloxane as DC5225C or DC5185, PEG-9 Dimethicone, available as KF6017 or KF6028 from Shin-Etsu, cetyl dimethicone copolyol-polyglyceryl-4-isostearate-hexylaurate available as ABIL® WE 09 from Goldschmidt Chemical Corporation, Hopewell, Va., Cetyl Dimethicone Copolyol (ABIL® EM 90), (ABIL® EM 97), Laurylmethicone Copolyol (5200), Cyclomethicone (and) Dimethicone Copolyol available as DC 3225 C from Dow Corning, and Cyclopentasiloxane and Dimethicone Copolyol available as GE SF 1528 from GE Silicones.

The compositions according to the invention may comprise the organosilicone emulsifiers in an amount preferably ranging from 2% to 15%, preferably from 5% to 12%, and better still from 7% to 10% by weight, relative to the total weight of the composition, and including all ranges and sub ranges there between.

Additional Coloring Agent (Optional)

The cosmetic compositions disclosed herein may further comprise at least one coloring agent.

Suitable coloring agents in accordance with various exemplary embodiments of the disclosure can produce, for instance, alone or as a mixture, a coloration having a yellow or orange hue. The coloring agents may, in certain embodiments, exhibit a significant reflectance in the range from about 550 to about 675 nm.

The at least one coloring agent may, for example, be chosen from mineral or organic pigments, coloring polymers, liposoluble dyes, organic lacquers, metallic powders, and mixtures thereof. A non-exhaustive list of suitable coloring agents can be found in the CTFA Cosmetic Ingredient Handbook, 11th Edition Cosmetic and Fragrance Association, Inc., Washington D.C. (2006).

It is within the ability of one skilled in the art to select the coloring agents and their amounts to adjust the color of the composition to create the desired effect, for example to adjust the bulk color of the composition and/or the color of the composition on the skin. Generally, the at least one coloring agent may be present in the composition in an amount ranging from about 5% to about 25% by weight, for example, from about 10% to about 20%, by weight, relative to the weight of the formula.

Volatile Solvents (Optional)

The emulsion, for example, may preferably comprise from 30% to 45% by weight, relative to the total weight of the emulsion, of oils, wherein said oils comprise at least one volatile hydrocarbon oil, at least one volatile oil, and at least one additional non-volatile oil, and, for example, from 30% to 40% by weight, relative to the total weight of the emulsion, of oils, wherein said oils comprise at least one volatile hydrocarbon oil, at least one volatile oil, and at least one additional non-volatile oil.

The cosmetic compositions disclosed herein may comprise at least one volatile solvent. As used herein, the term “volatile solvent” is intended to denote any non-aqueous solvent capable of evaporating on contact with the skin or a keratinous substrate in less than one hour, at approximately room temperature and atmospheric pressure. The at least one volatile solvent disclosed herein are liquid at room temperature, with a non-zero vapor pressure at room temperature and atmospheric pressure, ranging, for example, from 0.13 Pa to 40,000 Pa (10⁻³ to 300 mmHg), such as from 1.3 Pa to 13,000 Pa (0.01 to 100 mmHg), or from 1.3 Pa to 1,300 Pa (0.01 to 10 mmHg). The volatile oils may be chosen, for example, from hydrocarbon-based oils, silicone oils, fluoro oils, and mixtures thereof.

As used herein, the term “hydrocarbon-based oil” means oil mainly comprising hydrogen and carbon atoms and optionally oxygen, nitrogen, sulfur and/or phosphorus atoms. The volatile hydrocarbon-based oils may be chosen, for example, from hydrocarbon-based oils comprising from 8 to 16 carbon atoms, such as branched C₈-C₁₆ alkanes, for instance C₈-C₁₆ isoalkanes of petroleum origin (also known as isoparaffins), such as isododecane (also known as 2,2,4,4,6-pentamethylheptane), isoeicosane, isooctane, isodecane, and isohexadecane, for example, the oils sold under the trade names Isopar or Permethyl, branched C₈-C₁₆ esters and isohexyl neopentanoate, and mixtures thereof. Other volatile hydrocarbon-based oils, for instance petroleum distillates, such as those sold under the name Shell Solt by the company Shell, may also be used. The volatile solvent may be chosen, in various embodiments, from volatile hydrocarbon-based oils comprising from 8 to 16 carbon atoms, and mixtures thereof.

The volatile oils that may also be used include, for example, volatile silicones, for instance volatile linear or cyclic silicone oils, such as those with a viscosity 8 centistokes (8×10⁻⁵ m²/s) and, for example, comprising from 2 to 7 silicon atoms, these silicones optionally comprising at least one group chosen from alkyl and alkoxy groups comprising from 1 to 10 carbon atoms. Among the volatile silicone oils that may be used herein, mention may be made, for example, of octamethyl cyclotetrasiloxane, decamethyl cyclopentasiloxane, dodecamethyl cyclohexasiloxane, hexadecamethyl cyclohexasiloxane, heptamethyl hexyltrisiloxane, heptamethyl octyltrisiloxane, hexamethyl disiloxane, octamethyl trisiloxane, decamethyl tetrasiloxane, dodecamethyl pentasiloxane, and mixtures thereof. Volatile fluorinated solvents such as nonafluoromethoxybutane or perfluoromethylcyclopentane may, for example, also be used.

The volatile oil may be present in the cosmetic compositions in an amount ranging, for example, from about 1% to about 20% by weight, such as from about 2% to about 15% by weight, for example, from about 6% to about 14% by weight, relative to the total weight of the composition, including all ranges and subranges therebetween.

In various embodiments, the at least one volatile solvent is isododecane and is present in an amount ranging from about 2% to about 14 percent by weight, such as from about 8 percent to about 14 percent, or from about 12 percent to about 14% by weight, relative to the total weight of the cosmetic composition.

Non-Volatile Solvents (Optional)

The fatty phase of the emulsion disclosed herein may additionally comprise at least one additional non-volatile oil.

The at least one additional non-volatile oil may be present in an amount preferably ranging, for example, from 0.1% to 12% by weight, relative to the total weight of the emulsion, and further, for example, ranging from 1% to 5% by weight, relative to the total weight of the emulsion.

The at least one non-volatile oil additional oil may be chosen, for example, from synthetic ethers, hydrocarbon and silicone oils of mineral, animal, plant and synthetic origin, as long as they are compatible with the envisaged use.

According to this invention, suitable synthetic ether solvents include those containing from 10 to 40 carbon atoms, such as dicaprylyl ether.

The at least one non-volatile oil hydrocarbon oil may, for example, be chosen from paraffin oil, liquid paraffin, isoeicosane, mink oil, turtle oil, soya bean oil, perhydrosqualene, sweet almond oil, calophyllum oil, palm oil, grapeseed oil, sesame oil, maize oil, arara oil, rapeseed oil, sunflower oil, cottonseed oil, apricot oil, castor oil, avocado oil, jojoba oil, olive oil, and cereal germ oil; esters of lanolic acid, oleic acid, lauric acid and stearic acid; fatty esters, such as isopropyl myristate, isopropyl palmitate, butyl stearate, hexyl laurate, diisopropyl adipate, isononyl isononanoate, 2-ethylhexyl palmitate, 2-hexyldecyl laurate, 2-octyidecyl palmitate, 2-octyidodecyl myristate, 2-octyldodecyl lactate, 2-diethylhexyl succinate, diisostearyl malate, glycerine triisostearate, and diglycerine triisostearate; higher fatty acids such as myristic acid, palmitic acid, stearic acid, behenic acid, oleic acid, linoleic acid, linolenic acid, and isostearic acid; higher fatty alcohols such as cetanol, stearyl alcohol, oleyl alcohol, linoleyl alcohol, linolenyl alcohol, isostearyl alcohol, and octyldodecanol.

The at least one additional non-volatile oil silicone oil may, for example, be chosen from polysiloxanes modified with fatty acids, fatty alcohols, and polyoxyalkylenes; and polydimethylsiloxanes (PDMS), which may be optionally phenylated, such as phenyltrimethicones, and/or which may be optionally substituted with at least one group chosen from aliphatic and aromatic groups, and/or with at least one functional group, for example, chosen from hydroxyl, thiol and amine groups.

The fatty phase may further comprise at least one fatty substance chosen from waxes, gums and pasty fatty substances, wherein the pasty fatty substances may be chosen from pasty fatty substances of plant origin, animal origin, mineral origin, synthetic origin, and even silicone-based fatty substances.

The waxes solid at room temperature, which may be present in the emulsion disclosed herein, may, for example, be chosen from at least one of hydrocarbon waxes such as beeswax, Carnauba wax, Candelilla wax, Ouricoury wax, Japan wax, cork fibre waxes, sugarcane waxes, paraffin waxes, lignite waxes, microcrystalline waxes, lanolin wax, Montan wax, ozokerites, polyethylene waxes, waxes obtained by Fischer-Tropsch synthesis, hydrogenated oils, fatty esters and glycerides which are concrete at 25 degrees centigrade The waxes may also, for example, be chosen from silicone waxes, for example, silicone waxes chosen from alkyl, alkoxy and esters of polymethylsiloxane. The waxes may be provided in the form of stable dispersions of colloidal particles of wax as may be prepared according to known methods, such as those of “Microemulsions Theory and Practice”, L. M. Prince Ed., Academic Press (1977), pages 21-32. A wax which is liquid at room temperature, such as jojoba oil, may also be used in the emulsions disclosed herein.

The waxes may be present in an amount preferably ranging, for example, from 0.1% to 10% by weight, relative to the total weight of the emulsion, and further, for example, from 0.1% to 5% by weight, relative to the total weight of the emulsion.

Other Additives (Optional)

In addition, other cosmetic ingredients may be included in the compositions according to the disclosure. Such ingredients are known, and include but are not limited to humectants, antioxidants, surfactants, preservatives, fragrances, thickeners or texturizers, fatty compounds, emollients, coalescents, and/or plasticizers. One of skill in the art will be able to select appropriate types and amounts of additional cosmetic ingredients based on, for example, the type of cosmetic composition being formulated and the desired properties thereof, and taking care not harm the properties of the compositions. When present, such additional cosmetic ingredients may be present in the compositions according to the disclosure in a combined amount ranging from about 10% to about 80%, such as about 15% to about 60%, about 25% to about 40%, or about 30% to about 35%, including all ranges and subranges therebetween.

Water (Optional)

According to the invention, the water phase of the inventive compositions may comprise water in an amount preferably ranging from about 5% about 30%, preferably from about 10% to about 28%, most typically from about 15% about 25%, including all ranges and subranges therebetween, by weight, relative to the total weight of the compositions.

According to one embodiment of this invention the inventive composition is a water-in-oil emulsion containing less than about 30% of a water phase and more than about 70% of an oil phase, per weight, relative to the total weight of the composition.

Additional Solvents (Optional)

The compositions of the invention may further include additional solvents. In particular, the aqueous phase may include at least one organic solvent that is water-miscible. Non-limiting examples of suitable organic solvents include C₁₋₄ alkanols, such as ethanol, isopropanol, t-butyl alcohol; glycerol; glycols and glycol ethers such as 2-butoxyethanol, propylene glycol, butylene glycol, monomethyl ether of propylene glycol, monoethyl ether and monomethyl ether of diethylene glycol, aromatic alcohols such as benzyl alcohol and phenoxyethanol; analogous products and mixtures of the foregoing products.

The amount of the other solvents present in the inventive compositions is preferably from about 0.5% to about 10%, including all ranges and subranges therebetween, by weight, relative to the total weight of the compositions.

Cosmetic Method

The instant disclosure also relates to a cosmetic method for making up and/or enhancing the appearance of a keratinous substrate, particularly skin, the method comprising applying to the keratinous substrate a water-in-oil emulsion of the present invention. Preferably, the applied water-in-oil composition comprises:

from about 0.05% to about 80% of at least one hydrophobically treated powder;

from about 0.05% to about 15% of at least two mattifying powders;

from about 1% to about 30% of at least one siloxane resin;

from about 0.5% to about 20% of at least one oil absorbing powder;

optionally from about 0.5% to about 6% of at least one adhesive agent; and

optionally from about 1% to about 30% of at least one silicone elastomer, by weight, relative to the total weight of the composition.

All embodiments disclosed above with respect to the cosmetic compositions are equally applicable to the cosmetic method and are intended to fall within the scope of the disclosure.

The water-in-oil cosmetic composition according to the invention may be packed in a cosmetic container delimiting at least one compartment which comprises the cosmetic composition, the container being closed by a closing member.

Examples and Method of Making the Inventive Compositions

The following examples are intended to be non-restrictive and explanatory only, with the scope of the invention being defined by the claims.

TABLE 1
Preparation of Inventive Composition

| | | | Concen- |
|---|---|---|---|
| Phase | | Chemical Name | tration |
| Oil phase | A1 | PEG-10 DIMETHICONE | 2.75 |
| | A1 | CETYL PEG/PPG-10/1 | 0.9 |
| | | DIMETHICONE | |
| | A1 | ISODODECANE | 7 |
| | A1 | ISONONYL ISONONANOATE | 2.3 |
| | A1 | DICAPRYLYL ETHER | 2.5 |
| | A1 | POLYPROPYLSILSESQUIOXANE | 4.5 |
| | | (and) ISODODECANE | |
| | A2 | IRON OXIDES (and) | 4.9 |
| | | ACRYLATES/DIMETHICONE | |
| | | COPOLYMER | |
| | A2 | IRON OXIDES (and) | 0.85 |
| | | ACRYLATES/DIMETHICONE | |
| | | COPOLYMER | |
| | A2 | IRON OXIDES (and) | 0.7 |
| | | ACRYLATES/DIMETHICONE | |
| | | COPOLYMER | |
| | A2 | TITANIUM DIOXIDE | 8.55 |
| | A3 | DIMETHICONE/VINYL | 1 |
| | | DIMETHICONE | |
| | | CROSSPOLYMER | |
| | A4 | DISTEARDIMONIUM | 6 |
| | | HECTORITE (and) PROPYLENE | |
| | | CARBONATE | |
| | A5 | LAURYL PEG-9 | 2.75 |
| | | POLYDIMETHYLSILOXYETHYL | |
| | | DIMETHICONE | |
| | A5 | POLYGLYCERYL-4 | 0.9 |
| | | ISOSTEARATE | |
| | A6 | DIMETHICONE (and) | 11 |
| | | DIMETHICONE | |
| | | CROSSPOLYMER | |
| | A6 | TRIMETHYLSILOXYSILICATE | 2 |
| | | (and) | |
| | | POLYPROPYLSILSESQUIOXANE | |
| | A6 | METHYL METHACRYLATE | 2.3 |
| | | CROSSPOLYMER (and) | |
| | | MINERAL OIL | |
| | A6 | POLYLACTIC ACID | 2 |
| | A6 | POLYMETHYLSILSESQUIOXANE | 2 |
| | A6 | SILICA SILYLATE | 0.1 |
| | A6 | CALCIUM SODIUM | 3 |
| | | BOROSILICATE | |
| | A7 | ALCOHOL DENAT. | 4 |
| Water Phase | B1 | WATER | 23 |
| | B1 | GLYCERIN | 1.5 |
| | B1 | DIPROPYLENE GLYCOL | 1.5 |
| | B2 | HYDROXYETHYL | 0.5 |
| | | ACRYLATE/SODIUM | |
| | | ACRYLOYLDIMETHYL TAURATE | |
| | | COPOLYMER | |
| Incorporated | C | ALUMINUM STARCH | 1.5 |
| after | | OCTENYLSUCCINATE (and) | |
| emulsification | | ACRYLATES COPOLYMER (and) | |
| of phase A | | MAGNESIUM CARBONATE | |
| and B | | | |

In an embodiment the invention relates to a method of making the above-described a water-in-oil cosmetic composition which is made according to steps described below.

1. Ingredients of Phase A1 were combined in a main kettle/beaker,

2. Then, all compounds of Phase A2 were added in the main kettle and all ingredients were homogenized for 2 hours using a Silverston mixer. In order to maintain the room temperature of the batch, the kettle was placed in an ice bath.

3. Next, the compounds of Phase A3 were added and mixed for additional 5 minutes and then all ingredient of Phase A4 added and grinded for an additional 10-15 minutes. The ice bath was still used to keep the temperature of the formulation at the room temp.

4. In a side kettle/beaker combined ingredients of phase B, mixed them until homogenous and clear mixture was obtained.

5. The composition from the main kettle was moved to Rayneri mixer, added with ingredients of Phase A5 and cut with a chopper blade until the mixture was homogeneous.

6. Finally, the ingredients of Phase 6 were separately added and mixed until the composition had a homogenous texture. After that Phase A7 was incorporated

7. With both phases at room temp added in phase B and continue mixing for 10-15 minutes using a homogenizing blade.

8. Then added phase C and continue mixing until homogenous using the chopper blade for about 5 minutes. In order to maintain the room temperature of the prepared batch, all steps from 1-8 were done in an ice bath.

The resulting compositions were smooth and creamy, easy to apply and afforded even application. The compositions provided a nice matte finish, good oil control, evened skin tone and were not ashy on darker skin shades.

TABLE 2
Examples of Inventive Compositions

| Trade | | BLEND | Inv | Inv. | Inv | Inv. | Inv. |
|---|---|---|---|---|---|---|---|
| Name | INCI US | BREAKDOWN | 1 | 2 | 3 | 4 | 5 |
| ASC-2 | IRON OXIDES (and) | Iron Oxides/CI | 0.63 | 0.63 | 0.63 | 0.63 | 0.63 |
| BLACK BL | ACRYLATES/DIMETHICONE | 77499 98%; | | | | | |
| 100P | COPOLYMER | Acrylates/Dimethicone | | | | | |
| | | Copolymer 2% | | | | | |
| ASC-5 | IRON OXIDES (and) | Iron Oxides/CI | 0.86 | 0.86 | 0.86 | 0.86 | 0.86 |
| RED R- | ACRYLATES/DIMETHICONE | 77491 95%; | | | | | |
| 516P | COPOLYMER | Acrylates/Dimethicone | | | | | |
| | | Copolymer 5% | | | | | |
| ASC-7 | IRON OXIDES (and) | Iron Oxides/CI | 4.32 | 4.32 | 4.32 | 4.32 | 4.32 |
| YELLOW | ACRYLATES/DIMETHICONE | 77492 93%; | | | | | |
| LL-100P | COPOLYMER | Acrylates/Dimethicone | | | | | |
| | | Copolymer 7% | | | | | |
| ASC-2 | TITANIUM DIOXIDE | Iron Oxides/CI | 9.19 | 9.19 | 9.19 | 9.19 | 9.19 |
| TIO2 CR- | | 77891 95.10%; | | | | | |
| 50 | | Aluminum Hydroxide 2.9%; | | | | | |
| | | Acrylates/Dimethicone | | | | | |
| | | Copolymer 2% | | | | | |
| WICKENOL | ISONONYL | | 0.7 | 0.7 | 0.7 | 0.7 | 0.7 |
| 151 | ISONONANOATE | | | | | | |
| CETIOL OE | DICAPRYLYL ETHER | | 2.5 | 2.5 | 2.5 | 2.5 | 2.5 |
| LUXSIL | CALCIUM SODIUM | | 2 | 2.5 | 2 | 2 | 1.5 |
| | BOROSILICATE | | | | | | |
| BENTONE | DISTEARDIMONIUM | Isododecane 87%; | 6 | 6 | 6 | 6 | 6 |
| GEL ISD-V | HECTORITE (and) | Disteardimonium | | | | | |
| | PROPYLENE | Hectorite 10%; | | | | | |
| | CARBONATE | Propylene Glycol 3% | | | | | |
| AEROGEL | SILICA SILYLATE | | 0.05 | 0.1 | 0.05 | 0.05 | 0.05 |
| V-220 | | | | | | | |
| TOSPEARL | POLYMETHYL- | | | | 1 | 1.5 | 1 |
| 150-KA | SILSESQUIOXANE | | | | | | |
| NATRASORB | ALUMINUM STARCH | Aluminum Starch | 2 | 2.45 | 2 | 2 | 2 |
| HFB | OCTENYLSUCCINATE | Octenylsuccinate | | | | | |
| | (and) ACRYLATES | 85%; Acrylates | | | | | |
| | COPOLYMER (and) | Copolymer 10%; | | | | | |
| | MAGNESIUM CARBONATE | Magnesium Carbonate 5% | | | | | |
| SEPINOV | HYDROXYETHYL | Hydroxyethyl | 0.5 | 0.5 | 0.5 | 0.5 | 0.5 |
| EMT 10 | ACRYLATE/SODIUM | Acrylate/Sodium | | | | | |
| | ACRYLOYLDIMETHYL | Acrylolydimethyl | | | | | |
| | TAURATE COPOLYMER | Taurate 90%; | | | | | |
| | | Water 5%, | | | | | |
| | | Sorbitan Isostearate 2.5%, | | | | | |
| | | Polysorbate 60 2.5% | | | | | |
| SEPIMAT | METHYL METHACRYLATE | Methyl Methacrylate | 1.5 | 1 | 1 | 0.5 | 1 |
| H-10 | CROSSPOLYMER | Crosspolymer 81%; | | | | | |
| | (and) MINERAL OIL | Mineral Oil 19% | | | | | |
| TORAY | POLYLACTIC ACID | | 3 | 2.5 | 2.5 | 2.5 | 3 |
| PLA | | | | | | | |
| DC 556 | PHENYL | | 2.6 | 2.6 | 2.6 | 2.6 | 2.6 |
| FLUID | TRIMETHICONE | | | | | | |
| | CETYL PEG/PPG-10/1 | | 0.9 | 0.9 | 0.9 | 0.9 | 0.9 |
| | DIMETHICONE | | | | | | |
| DC 9506 | DIMETHICONE/VINYL | | 1 | 1 | 1 | 1 | 1 |
| POWDER | DIMETHICONE | | | | | | |
| | CROSSPOLYMER | | | | | | |
| KF-6017 | PEG-10 DIMETHICONE | | 2.75 | 2.75 | 2.75 | 2.75 | 2.75 |
| DC 9041 | DIMETHICONE (and) | Dimethicone/Vinyl | 11 | 11 | 11 | 11 | 11 |
| SILICONE | DIMETHICONE | Dimethicone | | | | | |
| ELASTOMER | CROSSPOLYMER | Crosspolymer 98%; | | | | | |
| BLEND | | Laureth-4 2% | | | | | |
| KF-6038 | LAURYL PEG-9 | | 2.75 | 2.75 | 2.75 | 2.75 | 2.75 |
| | POLYDIMETHYLSILOXYETHYL | | | | | | |
| | DIMETHICONE | | | | | | |
| DC 680 ID | POLYPROPYL- | Polypropyl- | 4.5 | 4.5 | 4.5 | 4.5 | 4.5 |
| FLUID | SILSESQUIOXANE (and) | silsesquioxane 72%; | | | | | |
| | ISODODECANE | Isododecane 28% | | | | | |
| DC MQ- | TRIMETHYLSILOXY- | Trimethylsiloxy- | 2 | 2 | 2 | 2 | 2 |
| 1640FLAKE | SILICATE (and) | silicate 65%; | | | | | |
| RESIN | POLYPROPYLSILSES- | Polypropyl- | | | | | |
| | QUIOXANE | silsesquioxane 35% | | | | | |
| DIPROPYLENE | DIPROPYLENE GLYCOL | | 1.5 | 1.5 | 1.5 | 1.5 | 1.5 |
| GLYCOL | | | | | | | |
| | ALCOHOL DENAT. | | 4 | 4 | 4 | 4 | 4 |
| DI WATER | WATER | | 23 | 23 | 23 | 23 | 23 |
| GLYCERIN | GLYCERIN | | 1.5 | 1.5 | 1.5 | 1.5 | 1.5 |
| PERMETHYL | ISODODECANE | | 8.35 | 8.35 | 8.35 | 8.35 | 8.35 |
| 99A | | | | | | | |
| ISOLAN GI | POLYGLYCERYL-4 | | 0.9 | 0.9 | 0.9 | 0.9 | 0.9 |
| 34 | ISOSTEARATE | | | | | | |

Methods of Testing

The inventive compositions were tested to compare mattity and wear of color against a Comparator A having a structure as disclosed below.

Comparator A: water, cyclopentasiloxane, polymethyl methacrylate, butylene glycol, ethylhexyl methoxycinnamate, peg-10 dimethicone, trimethylsiloxysilicate, xanthan gum, dimethicone, magnesium sulfate, dimethicone/peg-10/15 crosspolymer, dimethicone/peg-10 crosspolymer, dimethicone crosspolymer, silica, botanical extracts, pigments, fillers.

Test Methods

WEAR OF MATTITY—Maintenance of matitty throughout wear from initial application to end of wear

COLOR: WEAR OF COLOR—maintenance of color throughout wear from initial application to end of wear

PROCEDURE:

1. CHOOSE PARTICIPANTS (minimum 5)

2. INITIAL APPLICATION IN THE MORNING
A. HALF FACE APPLICATION
   i. Comparator A on one side
   ii. Invention composition on the other side

3. TAKE PICTURES
A. BARE FACE
B. IMMEDIATELY AFTER APPLICATION
C. ON DRYDOWN
D. AFTER FOUR HOURS OF WEAR

4. PARTICIPANT WEARS PRODUCTS FOR FOUR HOURS
A. RETURNS TO LAB AFTER 4 HOURS (End of wear) FOR PICTURES
B. EVALUATION OF PICTURES
C. VISUALLY COMPARE PICTURES AND NOTE DIFFERENCES & CHANGES FROM INTIAL TO 4 HOUR WEAR
   i. MATTITY
   ii. COLOR

ASSESSMENTS & RATINGS

| COSMETIC | Comparator A | | Inventive Composition 1 | | Inventive Composition 2 | |
|---|---|---|---|---|---|---|
| PROPERTY | — | RATING | | RATING | | RATING |

MATTITY

| | | | | | | |
|---|---|---|---|---|---|---|
| IMMEDIATE | LUMINOUS | 1.5 | MATTE | 4.4 | DEMI- | 4.2 |
| | | | | | MATTE: | |
| AT DRYDOWN | SEMI- | 2.3 | MATTE | 4.7 | MATTE | 4.4 |
| | MATTE | | | | | |
| AT 4 HOURS | SEMI- | 2.1 | MATTE | 4.4 | DEMI- | 4 |
| (Wear of Matitty) | MATTE | | | | MATTE | |
| SHINE/NO | | | | | | |
| MATITTY = 1; | | | | | | |
| VERY MATTE/NO | | | | | | |
| SHINE = 5 | | | | | | |

WEAR OF COLOR

| | | | | | | |
|---|---|---|---|---|---|---|
| IMMEDIATE | EVEN, | 5 | EVEN, FULL | 5 | EVEN FULL | 5 |
| | MEDIUM | | COVERAGE | | COVERAGE | |
| | COVERAGE | | | | | |
| AT DRYDOWN | EVEN, | 5 | EVEN, FULL | 5 | EVEN FULL | 5 |
| | MEDIUM | | COVERAGE | | COVERAGE | |
| | COVERAGE | | | | | |
| AT 4 HOURS | Faded in | 3 | EVEN, | 4 | EVEN, | 4 |
| | some areas | | MAINTAINED | | MAINTAINED | |
| | (blotchy) | | COLOR | | COLOR | |

CHANGE OF COLOR

| | | | | | | |
|---|---|---|---|---|---|---|
| IMMEDIATE | APPEARS | 5 | CONSISTENT | 5 | CONSISTENT | 5 |
| | LIGHTER | | WITH | | WITH OTHER | |
| | LESS RED | | OTHER TIME | | TIME POINTS | |
| | (COMPARED | | POINTS | | | |
| | TO OTHER | | | | | |
| | TIME POINTS) | | | | | |
| AT DRYDOWN | REDDER | 3 | CONSISTENT | 4.7 | CONSISTENT | 4.7 |
| | | | WITH INITIAL | | WITH INITIAL | |
| AT 4 HOURS | REDDER, | 2 | CONSISTENT | 4.5 | CONSISTENT | 4.5 |
| | DIRTIER | | WITH INITIAL | | WITH INITIAL | |

DEFINITIONS:
MATITTY (RATING RANGE)
MATTE (4.01-5): CHARACTERIZED BY NO VISIBLE SHINE. NON REFLECTING. VISUAL POWDERY APPEARANCE
DEMI-MATTE(2.51-4.0): NOT A COMPLETELY POWDER APPEARANCE BUT NOT SHINY EITHER
SEMI-MATTE(2.01-2.5): CHARACTERIZED BY SLIGHT SHINE
LUMINOUS (1-2): CHARACTERIZED BY A DEWY APPEARANCE; SHINE APPEARANT ON SKIN
MATITTY: CHARACTERIZED BY A FLAT APPEARANCE WITHOUT SHINE. DEGREE OF MATITTY CAN VARY. VARIATIONS OF MATTITY APPLY WITH DIFFERENTIATIONS RANGING FROM EXTREME MATTITY (RATING 5) TO NOT MATTE (RATING 1) (SEE ABOVE)
CHANGE OF COLOR: COLOR DIFFERENCE BETWEEN THE TIME POINTS; FROM INITIAL APPLICATION TO COLOR AT DRYDOWN TO SHADE AFTER 4 HOURS OF WEAR
WEAR OF COLOR: INDICATES HOW WELL THE COLOR WORE THROUGHOUT WEAR PERIOD. HOW MUCH PRODUCT REMAINS AND HOW EVEN IT IS ON SKIN
CONCLUSION:
Invention 1 imparts significantly matte appearance upon application and which is enhanced further on drydown. At four hours of wear it still gives some mattity with consistent wear of color throughout the entire wear period.
Invention 2 imparts a slightly matte appearance upon application and which is enhanced further on drydown. At four hours of wear it still gives mattity (comparable to comparator A) with consistent wear of color throughout the entire wear period.

What is claimed is:

1. A water-in-oil emulsion comprising:
a) at least one hydrophobically treated powder
b) at least two mattifying powders;
c) at least one silicone resin
d) at least one oil absorbing powder;
e) optionally at least one adhesive agent; and
f) optionally at least one silicone elastomer.

2. The water-in-oil emulsion of claim 1, wherein the at least one hydrophobically treated powder is treated at the surface with a hydrophobic agent selected from the group consisting of silanes, silicones, fatty acid soaps, C₉₋₁₅ fluoroalcohol phosphates, acrylate/dimethicone copolymers, mixed C₉-C₁₅ fluoroalcohol phosphate/silicone copolymers, lecithins, hydrogenated lecithin, carnauba wax, polyethylene, chitosan, optionally acylated amino acids, and mixtures thereof.

3. The water-in-oil emulsion of claim 1, wherein the at least one hydrophobically treated powder is present in an amount from about 0.05% to about 80% by weight, relative to the total weight of the emulsion.

4. The water-in-oil emulsion of claim 1, wherein the at least two mattifying powders are selected from the group consisting of polylactic acid powders, acrylic polymer powder, and mixtures thereof.

5. The water-in-oil emulsion of claim 1, wherein the at least two mattifying powders are present in an amount from about 0.05% to about 15% by weight, relative to the total weight of the emulsion.

6. The water-in-oil emulsion of claim 1, wherein the at least one silicone resin is selected from the group consisting of MQ resins, T propyl silicone resins, MQ-T propyl silicone resins, and mixtures thereof.

7. The water-in-oil emulsion of claim 1, wherein the at least one silicone resin is present in an amount from about 1% to about 30% by weight, relative to the total weight of the emulsion.

8. The water-in-oil emulsion of claim 1, wherein the at least one oil absorbing powder is selected from the group consisting of silica powder, talc, polyamide particles, polyethylene powders, and mixtures thereof.

9. The water-in-oil emulsion of claim 1, wherein the at least one oil absorbing powder is present in an amount from about 0.5% to about 20% by weight, relative to the total weight of the emulsion.

10. The water-in-oil emulsion of claim 1, wherein the emulsion comprises at least one silsesquioxane powder or at least one polymethylsilsesquioxane powders.

11. The water-in-oil emulsion of claim 1, wherein the at least one adhesive agent is present the emulsion in an amount from about 0.5% to about 6% by weight, relative to the total weight of the emulsion.

12. The water-in-oil emulsion of claim 1, wherein the at least one silicone elastomer is a non-emulsifying silicone elastomer.

13. The water-in-oil emulsion of claim 1, wherein the at least one silicone elastomer is present in the emulsion in an amount from about 1% to about 30% by weight, relative to the total weight of the emulsion.

14. The water-in-oil emulsion of claim 1, wherein the weight ratio of a first mattifying powder to a second mattifying powder is greater than about 2:1 and lower than about 1:10.

15. The water-in-oil emulsion of claim 1, wherein the ratio of the water phase to hydrophobically treated powder is lower than about 4:1.

16. A method of making up skin comprising applying the emulsion of claim 1 to skin.
